(19) United States
(12) Patent Application Publication
(10) Pub. No.: US 2025/0329803 A1
(43) Pub. Date: Oct. 23, 2025

(54) BATTERY DISCHARGE MECHANISM FOR SURGICAL INSTRUMENTS

(71) Applicant: Covidien LP, Mansfield, MA (US)

(72) Inventors: Lan CHANG, Shanghai (CN); Xingrui CHEN, Shanghai (CN); Anthony D. CALDERONI, North Haven, CT (US); John M. PANTAZIS, North Haven, CT (US); Eric WIGFORSS, North Haven, CT (US)

(73) Assignee: Covidien LP, Mansfield, MA (US)

(21) Appl. No.: 18/864,777

(22) PCT Filed: May 9, 2023
(86) PCT No.: PCT/CN2023/092921

§ 371 Date: Nov. 11, 2024

Related U.S. Application Data
(60) Provisional application No. 63/341,005, filed on May 12, 2022.

(51) Int. Cl. H01M 10/42; A61B 17/00; A61B 17/29; H01M 50/213; H01M 50/247

(52) U.S. Cl. CPC H01M 10/425; A61B 17/2909; H01M 50/213; H01M 50/247; A61B 2017/00734; H01M 2220/30

(57) ABSTRACT

A surgical instrument includes a handle assembly and a battery assembly configured to removably couple to the handle assembly. The handle assembly includes a handle housing, an electronic component within the housing, a handle finger extending from the handle housing, and an electrical contact electrically coupled to the electronic component. The battery assembly includes a battery housing, a battery disposed in the battery housing, a battery contact electrically coupled to the battery, and a discharge circuit board. The discharge circuit board is configured to discharge the battery and is movable within the battery housing from a first position to a second position by the handle finger upon coupling the battery assembly to the handle assembly.

### CROSS-REFERENCE TO RELATED APPLICATIONS

This application claims the benefit of, and priority to, U.S. Provisional Patent Application Ser. No. 63/341,005, filed on May 12, 2022, the entire contents of which being incorporated by reference herein.

### BACKGROUND

### Technical Description

The present disclosure relates to powered surgical instruments. More specifically, the present disclosure relates to a battery discharge mechanism for electromechanical, hand-held surgical instruments.

### Background of Related Art

Powered surgical instruments utilize one or more electrical components, such as circuit boards, sensors, motors, etc. to operate various functions of the instrument. Handheld powered surgical instruments may have a corded power supply configuration or may be battery operated. Some battery-operated surgical instruments are powered by a removable battery pack, which is typically discarded after use. Many governing bodies across the globe require batteries to be fully discharged by the time they reach the end of their waste stream. Accordingly, a need exists for a low-cost mechanism that ensures the full discharge of a battery subsequent the battery's use and prior to disposal.

### SUMMARY

The present disclosure provides a surgical instrument. The surgical instrument includes a handle assembly and a battery assembly configured to removably couple to the handle assembly. The handle assembly includes a handle housing, an electronic component within the housing, a handle finger extending from the handle housing, and an electrical contact electrically coupled to the electronic component. The battery assembly includes a battery housing, a battery disposed in the battery housing, a battery contact electrically coupled to the battery, and a discharge circuit board. The discharge circuit board is configured to discharge the battery and is movable within the battery housing from a first position to a second position by the handle finger upon coupling the battery assembly to the handle assembly.

In an aspect, the surgical instrument includes an elongated shaft extending from the handle assembly and an end effector operably coupled to the elongated shaft.

In an aspect, the battery assembly includes a spring clip configured to maintain the discharge circuit board in the second position after the battery assembly is removed from the handle assembly. Additionally, or alternatively, the spring clip may be configured to maintain the discharge circuit board in the first position prior to the battery assembly being coupled to the handle assembly.

In an aspect, the battery contact is configured to conductively contact a contact pad of the discharge circuit board when the battery assembly is removed from the handle assembly.

In an aspect, the handle finger includes a protrusion configured to move a portion of the battery contact to block conductive contact between the battery contact and the discharge circuit board when the battery assembly is coupled to the handle assembly.

In an aspect, the battery contact is configured to conductively contact the electrical contact of the handle assembly when the battery assembly is coupled to the handle assembly to electrically couple the battery to the electronic component.

In an aspect, the handle finger defines an opening and the electrical contact extends through the opening.

In an aspect, the discharge circuit board is configured to longitudinally slide along a track of the battery assembly.

In an aspect, the battery housing includes a battery connector and the handle housing includes a handle connector configured to releasably mate with the battery connector.

In an aspect, the handle finger includes a first protrusion and a second protrusion. The first protrusion is configured to move the discharge circuit board from the first position to the second position when the battery assembly is coupled to the handle assembly. The second protrusion is configured to move a portion of the battery contact to block conductive contact between the battery contact and the discharge circuit board.

In an aspect, the battery contact is a first battery contact electrically coupled to a first terminal of the battery cell and configured to contact a first contact pad of the discharge circuit board when the discharge circuit board is in the second position. Additionally, or alternatively, the battery assembly further includes a second battery contact electrically coupled to a second terminal of the battery cell and configured to contact a second contact pad of the discharge circuit board when the battery assembly is in the second position and in a discharge state.

In another aspect of the disclosure, a battery assembly is provided. The battery assembly includes a battery housing, a battery disposed within the battery housing, a battery contact electrically coupled to the battery, and a discharge circuit board including a contact pad and movable within the battery housing between a first position in the battery housing and a second position in the battery housing, wherein the discharge circuit board is configured to discharge the battery cell when the contact pad is electrically coupled to the battery contact.

In an aspect, the battery assembly includes a spring clip configured to maintain the discharge circuit board in the first position and the second position.

In an aspect, the battery assembly includes a track defined in the battery housing, wherein the discharge circuit board is configured to longitudinally slide along the track.

In an aspect, the battery contact is a second battery contact electrically coupled to a second terminal of the battery cell and configured to contact a second contact pad of the discharge circuit board when the discharge circuit board is in the second position and in a discharge state. Additionally, or alternatively, the battery assembly further includes a first battery contact configured to contact a first contact pad of the discharge circuit board when the discharge circuit board is in the second position.

In another aspect of the disclosure, a surgical instrument is provided. The surgical instrument includes a handle assembly and a battery assembly configured to removably couple to the handle assembly. The handle assembly includes a handle housing, an electronic component disposed within the handle housing, and an electrical contact electrically coupled to the electronic component. The battery assembly includes a battery housing, a battery disposed within the battery housing, a battery contact electrically coupled to the battery, and a discharge circuit board including a contact pad and movable within the battery housing between a first position in the battery housing and a second position in the battery housing, wherein the discharge circuit board is configured to discharge the battery cell when the contact pad is electrically coupled to the battery contact.

In an aspect, the battery assembly includes a spring clip configured to maintain the discharge circuit board in the first position prior to the battery assembly being coupled to the handle assembly and maintain the discharge circuit board in the second position after the battery assembly is removed from the handle assembly.

In an aspect, the battery contact is configured to conductively contact a contact pad of the discharge circuit board when the battery assembly is removed from the handle assembly.

In an aspect, the handle assembly includes a handle finger and a protrusion extending from the handle finger, the protrusion configured to move a portion of the battery contact to block conductive contact between the battery contact and the discharge circuit board when the battery assembly is coupled to the handle assembly.

In an aspect, the handle assembly includes a handle finger defining an opening and the electrical contact extends through the opening.

In an aspect, the battery contact is configured to conductively contact the electrical contact of the handle assembly when the battery assembly is coupled to the handle assembly to electrically couple the battery to the electronic component.

In an aspect, the discharge circuit board is configured to longitudinally slide along a track of the battery assembly.

In an aspect, the handle assembly includes a handle finger which has a first protrusion and a second protrusion. The first protrusion is configured to move the discharge circuit board from the first position to the second position when the battery assembly is coupled to the handle assembly. The second protrusion is configured to move a portion of the battery contact to block conductive contact between the battery contact and the discharge circuit board.

In an aspect, the battery contact is a second battery contact electrically coupled to a second terminal of the battery cell and configured to contact a second contact pad of the discharge circuit board when the discharge circuit board is in the second position and in a discharge state. Additionally, or alternatively, the battery assembly further includes a first battery contact configured to contact a first contact pad of the discharge circuit board when the discharge circuit board is in the second position.

### BRIEF DESCRIPTION OF THE DRAWINGS

Various aspects of the disclosed surgical instrument are described herein below with reference to the drawings, wherein:

FIG. 1 is a perspective view of a powered surgical instrument including a handle assembly and a battery assembly according to an embodiment of the present disclosure;

FIG. 2 is a side perspective view of a first cross-section of the handle assembly and the battery assembly of the powered surgical instrument of FIG. 1 in a pre-installation state;

FIG. 3 is a side perspective view of a second cross-section of the handle assembly and the battery assembly of the powered surgical instrument of FIG. 1 in the pre-installation state;

FIG. 4 is a side perspective view of a third cross-section of the handle assembly and the battery assembly of the powered surgical instrument of FIG. 1 in a post-installation state;

FIG. 5 is a side perspective view of the third cross-section of the handle assembly and the battery assembly of the powered surgical instrument of FIG. 1 in a disposal state;

FIG. 6 is a side perspective view of a fourth cross-section of the handle assembly and the battery assembly of the powered surgical instrument of FIG. 1 in the disposal state;

FIG. 7 is a rear perspective view of the handle assembly and the battery assembly of the powered surgical instrument of FIG. 1, with parts removed, in the disposal state;

FIG. 8 is a perspective view of a powered surgical instrument including a handle assembly and a battery assembly according to another embodiment of the present disclosure;

FIG. 9 is a perspective view of the powered surgical instrument of FIG. 8 with the battery assembly separated from the handle assembly;

FIG. 10 is a rear perspective view of the handle assembly of the powered surgical instrument of FIG. 8;

FIG. 11 is a rear perspective view of the handle assembly of the powered surgical instrument of FIG. 8 with a handle housing of the handle assembly shown in phantom to reveal the internal components of the handle assembly;

FIG. 12 is a front perspective view of the battery assembly of the powered surgical instrument of FIG. 8;

FIG. 13 is an exploded view of the battery assembly of the powered surgical instrument of FIG. 8 with parts separated;

FIG. 14 is a perspective view of the battery assembly of the powered surgical instrument of FIG. 8 with parts separated;

FIG. 15 is a rear perspective view of a cover of the battery assembly of the powered surgical instrument of FIG. 8;

FIG. 16 is a rear perspective view, from a first side, of a discharge circuit board of the battery assembly of the powered surgical instrument of FIG. 8;

FIG. 17 is a rear perspective view, from a second side, of a discharge circuit board of the battery assembly of the powered surgical instrument of FIG. 8;

FIG. 18 is a rear perspective view of a portion of the handle assembly and the cover of the battery assembly separated from the handle assembly of the powered surgical instrument of FIG. 8 with the printed circuited board in a first position;

FIG. 19 is a rear perspective view, from a first side, of a portion of the handle assembly and the cover of the battery assembly connected to the handle assembly of the powered surgical instrument of FIG. 8 with the discharge circuit board in a second position;

FIG. 20 is a rear perspective view, from a second side, of a portion of the handle assembly and the cover of the battery assembly connected to the handle assembly of the powered surgical instrument of FIG. 8 with the discharge circuit board in a second position; and

FIG. 21 is a rear perspective view of the cover of the battery assembly of the powered surgical instrument of FIG. 8 with the discharge circuit board in a second position and discharge state, post removal of the battery assembly from the handle assembly.

### DETAILED DESCRIPTION

The disclosed powered surgical instruments are described in detail with reference to the drawings in which like reference numerals designate identical or corresponding elements in each of the several views. However, it is to be understood that the aspects of the disclosure described herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during usage of the device in a customary manner, while the term “distal” is used generally to refer to that portion of the device that is farther from the clinician during usage of the device in a customary manner. In addition, the term “endoscopic” is used generally to refer to endoscopic, laparoscopic, arthroscopic, and/or any other procedure conducted through small diameter incision or cannula. Further, the term “clinician” is used generally to refer to medical personnel including doctors, nurses, and support personnel.

Many governing bodies across the globe require batteries (e.g., lithium cells) to be fully discharged by the time they reach the end of their waste stream. Handheld surgical instruments can utilize removable battery packs to power the instrument during use in a procedure. If the battery pack is removed from the handheld device, thereby disconnecting the electrical load, prior to being fully discharged, the battery will not meet this requirement and can reach the end of its waste stream with electrical energy still available. The surgical instrument disclosed includes a mechanism for guaranteeing full discharge of the battery irrespective of whether the battery remains connected to the surgical instrument or is removed from the surgical instrument after use.

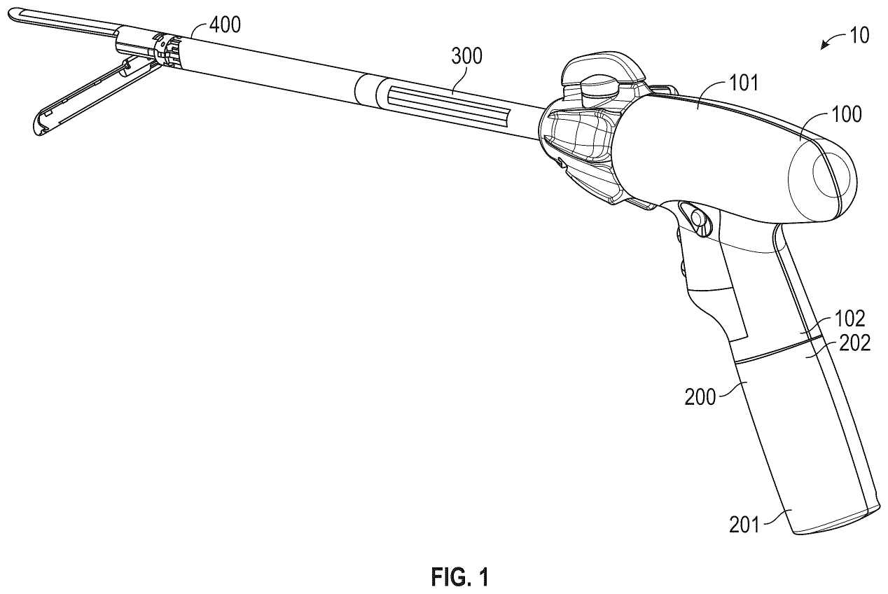

Referring initially to FIG. 1, an electromechanical, hand-held, powered surgical instrument 10, in accordance with aspects of the disclosure of the disclosure includes a handle assembly 100, an elongated shaft 300 extending from the handle assembly 100, and an end effector 400 coupled to a distal end of the elongated shaft 300. The end effector 400 is configured for actuation and manipulation by the handle assembly 100. Surgical instrument 10 may be any battery powered instrument, handheld or robotic, such as a powered surgical stapler, grasper, cutter, electrosurgical device, etc.

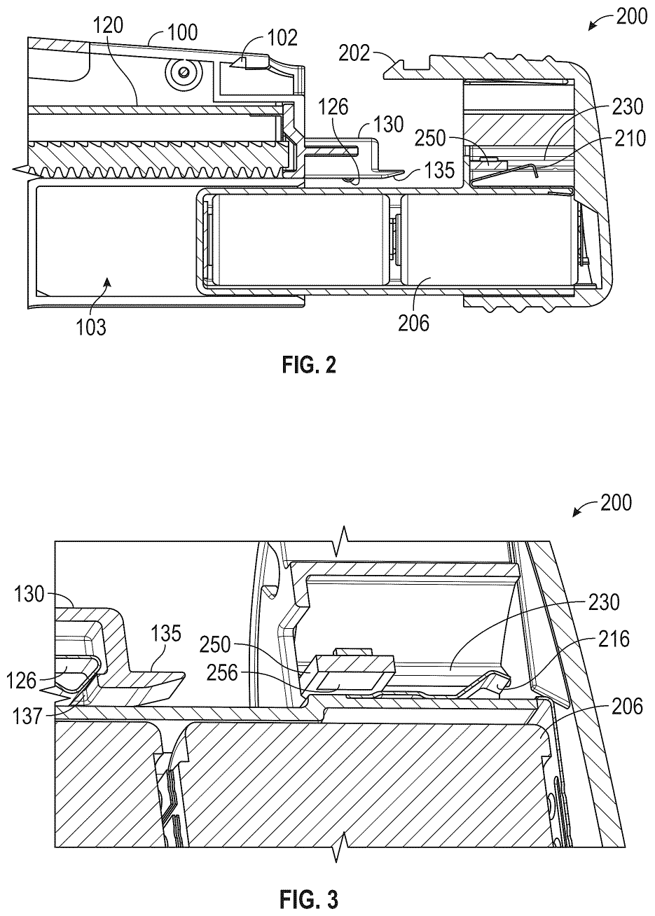

Handle assembly 100 includes a handle housing 101, which houses various electronic component(s) 120 (e.g., electronic component 120 circuit board, motors, sensors, controllers, etc. FIG. 2) and mechanical components configured to control operation of the surgical instrument 10. The surgical instrument 10 additionally includes a battery assembly 200 which is configured to removably couple to the handle assembly 100 and supply power to the electronic components of the surgical instrument 10. In an aspect, the handle housing 101 defines an opening 103 (FIG. 2) configured to receive at least a portion of the battery assembly 200 therein. In aspects, the handle assembly 100 includes a handle connector 102 which is configured to releasably engage a battery connector 202 formed by, or coupled to, a battery housing 201 of the battery assembly 200 to secure the battery assembly 200 to the handle housing 101. The handle connector 102 and battery connector 202 may be any suitable connection mechanism that enables releasable attachment of two components (e.g., press-clip, threading, etc.).

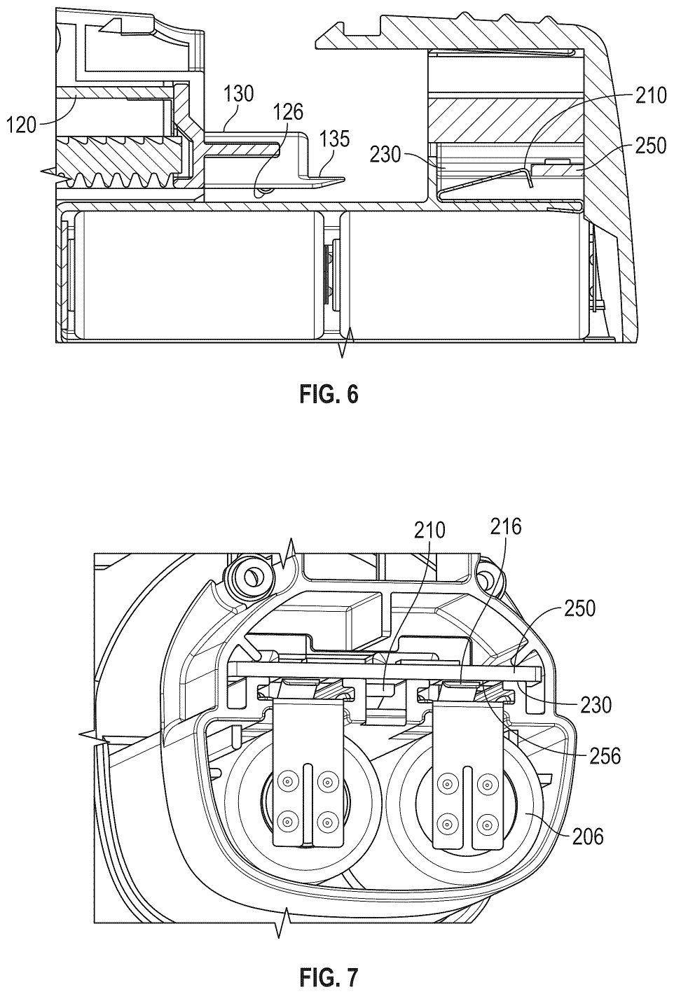

With reference to FIG. 2, the battery assembly 200 includes one or more battery cells 206 disposed within the battery housing 201, and a battery contact 216 conductively coupled to a terminal of the battery cell 206. The battery cell 206 is configured to electrically couple to the electronic component 120 of the surgical instrument 10 when the battery assembly 200 is coupled to the handle assembly 100 to form a circuit therewith and provide power to the electrical components (e.g., the electronic component 120, motors, sensors, etc.) of the surgical instrument 10. Only a single battery contact 216 is illustrated and described for brevity, but it is appreciated that the battery assembly 200 may include a plurality of battery contacts 216 or independent positive and negative terminal contacts (e.g., corresponding to positive and negative terminals of the battery cell 206, respectively). The battery assembly 200 also includes a discharge circuit board 250 which is configured to discharge the battery cell 206 after the battery cell 206 is used with the surgical instrument 10 (e.g., when the battery assembly 200 is in a disposal state, FIGS. 6-8), as described in further detail below.

The discharge circuit board 250 contains a passive resistive load which is conductively coupled to a contact pad 256 (FIGS. 5 and 7) and is movable within the battery housing 201 between a first position (FIGS. 2 and 3) and a second position. The passive resistive load may include one or more resistors. The contact pad 256 may be disposed on an underside of the discharge circuit board 250. In an aspect, the battery housing 201 defines a track 230 (FIGS. 3 and 7) within which the discharge circuit board 250 is configured to longitudinally slide between the first position and the second position. As with the battery contact 216, only a single contact pad 256 is illustrated and described for brevity, but it is appreciated that the discharge circuit board 250 may include two contact pads 256 or independent positive and negative terminal contacts (e.g., corresponding to positive and negative terminals of the battery cell 206, respectively).

The battery assembly 200 further includes a spring clip 210 or any other suitable retaining device that is positioned relative to the discharge circuit board 250 such that the spring clip 210 maintains the position of the discharge circuit board 250 in the first position prior to the battery assembly 200 ever being coupled to any handle assembly 100. The spring clip 210 also maintains the discharge circuit board 250 in the second position after the battery assembly 200 is removed from the handle assembly 100.

The handle assembly 100 includes a handle finger 130 extending from the handle housing 101 and a protrusion 135 extending from the handle finger 130. An electrical contact 126 is electrically coupled to the electronic component 120 and is configured to conductively contact a battery contact 216 of the battery assembly 200 to close the electrical circuit, thereby providing electrical power to the electronic components (e.g., electronic component 120) of the handle assembly 100 when the battery assembly 200 is coupled to the handle assembly 100. In an aspect, the electrical contact 126 extends through an opening 137 (FIG. 7) defined through the handle finger 130. Only a single electrical contact 126 is illustrated and described for brevity, but it is appreciated that the electronic component 120 may include two electrical contacts 126 or independent positive and negative terminal contacts (e.g., corresponding to positive and negative terminals of the battery cell 206, respectively).

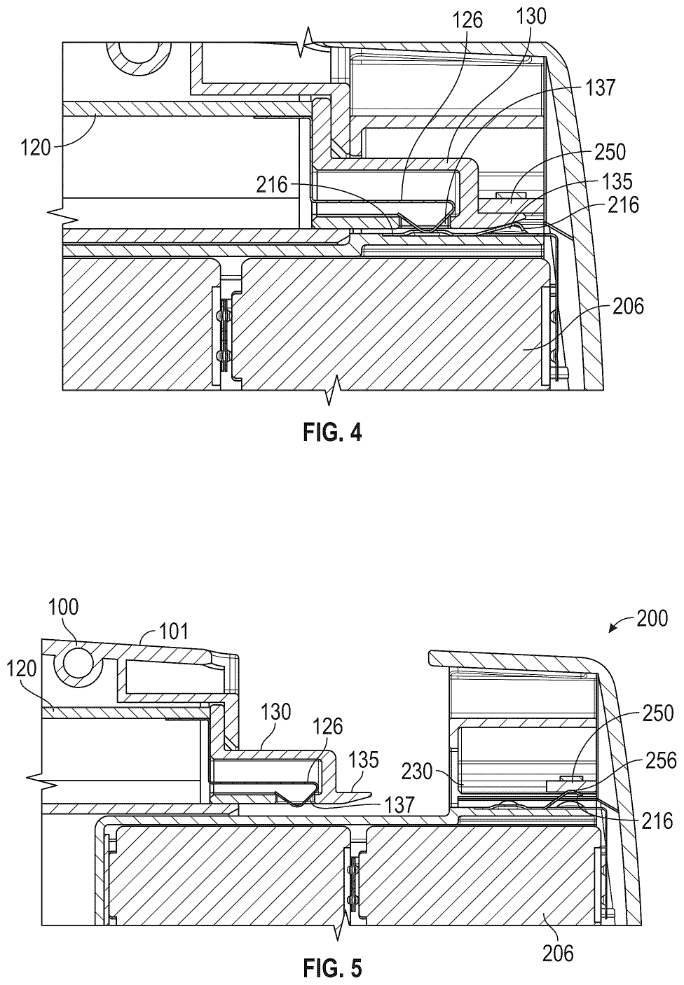

FIG. 3 illustrates the battery assembly 200 in a pre-installation state where, prior to the first use of the battery assembly 200, the discharge circuit board 250 is in the first position, and is maintained in this position by the spring clip 210. In the first position, the contact pad 256 is physically distanced from the battery contact 216, and therefore not in conductive contact therewith. After coupling the battery assembly 200 to the handle assembly 100, the battery assembly 200 is transitioned to a post-installation state (FIG. 4). During coupling of the battery assembly 200 to the handle assembly 100 (e.g., during the transition from the pre-installation state to the post-installation state), the handle finger 130 depresses (or otherwise disengages) the spring clip 210 and urges the discharge circuit board 250 from the first position toward the second position. In addition to urging the discharge circuit board 250 from the first position toward the second position, the protrusion 135 of the handle finger 130 slides under the discharge circuit board 250 to physically cover the contact pad 256 of the discharge circuit board 250, thereby preventing the contact pad 256 from being able to make conductive contact with the battery contact 216. In aspects, the battery contact 216 is moved (e.g., depressed) by the protrusion 135 to physically distance the battery contact 216 from the contact pad 256 in addition to, or in lieu of, the physical covering of the contact pad 256 by the protrusion 135. When the battery assembly 200 is in the post-installation state, the electrical contact 126 is electrically coupled to the battery contact 216, thereby forming the circuit and supplying power to the electronic component 120.

FIGS. 5-7 illustrate the battery assembly in a disposal state, that is, a state in which the battery assembly 200 has been coupled to, and then subsequently removed from, the handle assembly 100. In the disposal state, the discharge circuit board 250 is in the second position and is maintained in the second position by the spring clip 210. In addition, because the protrusion 135 of the handle finger 130 is no longer covering the contact pad 256 of the discharge circuit board 250, and no longer depressing the battery contact 216 to physically separate the battery contact 216 from the contact pad 256, the contact pad 256 is electrically coupled to the battery contact 216. When the contact pad 256 is electrically coupled to the battery contact 216 (e.g., when a positive terminal of the contact pad 256 is contacting a positive terminal of the battery contact 216 and/or a negative terminal of the contact pad 256 is contacting a negative terminal of the battery contact 216), a circuit is formed which enables the discharge circuit board 250 to discharge the battery cell 206. The discharge circuit board 250 will discharge the battery cell 206 until no more electrical energy is left in the battery cell 206 of the battery assembly 200.

The time to fully discharge the battery cell 206 of the battery assembly 200 depends on the capacity of the battery cell 206 and the resistive load of the discharge circuit board 250. In aspects, the resistive load of the discharge circuit board 250 may be selectable, manually or automatically, to control the discharge rate. Resistors forming the resistive load of the discharge circuit board 250 may be implemented in series and parallel to distribute electrical power as well as add safety redundancies to the discharging function of the discharge circuit board 250.

After being in the disposal state, if the battery assembly 200 is recoupled to the handle assembly 100, the protrusion 135 of the handle finger 130 will slide under the discharge circuit board 250 between the contact pad 256 and the battery contact 216 to break the conduction connection therebetween and enable reconnection of the electrical contact 126 to the battery contact 216.

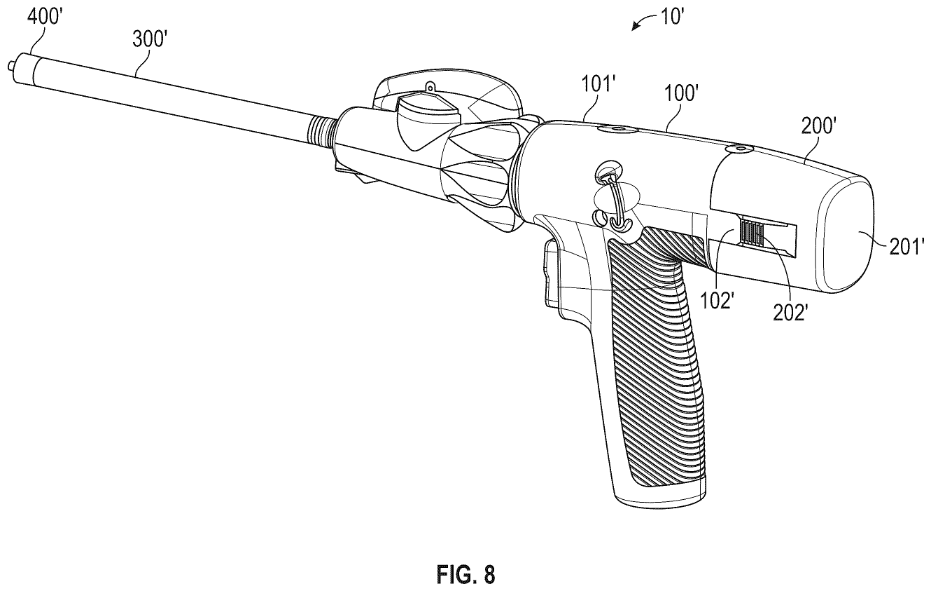

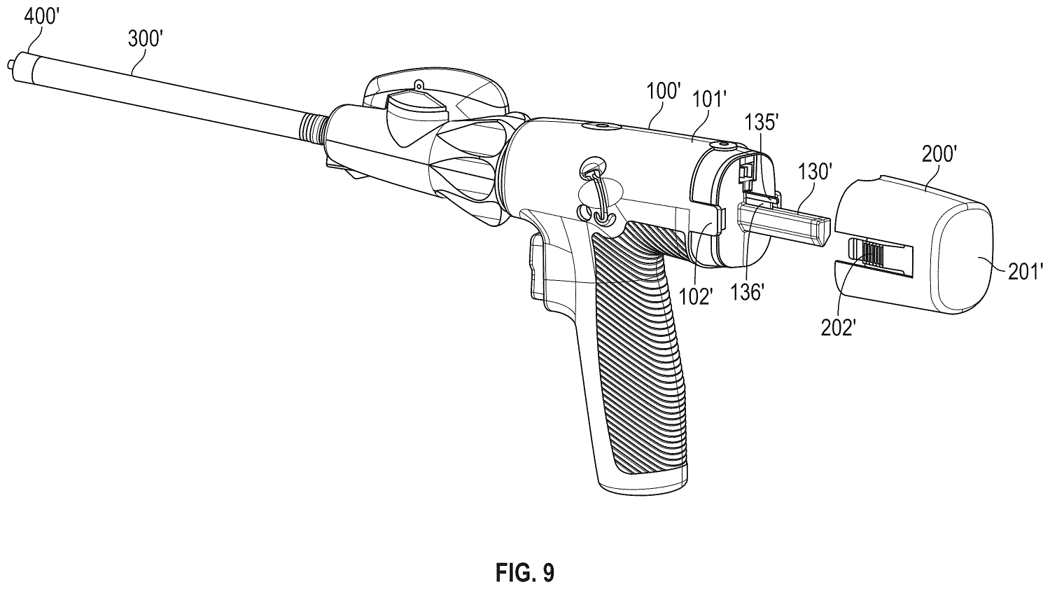

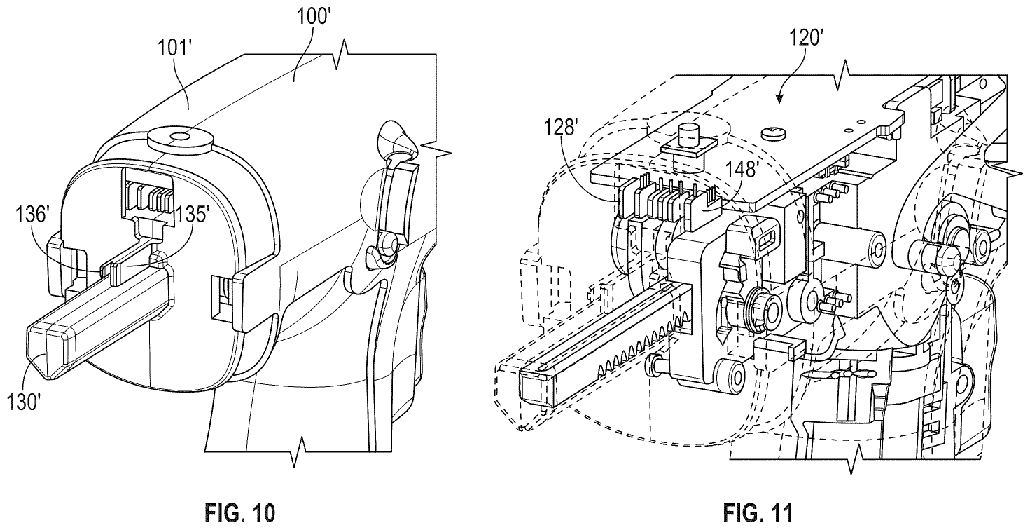

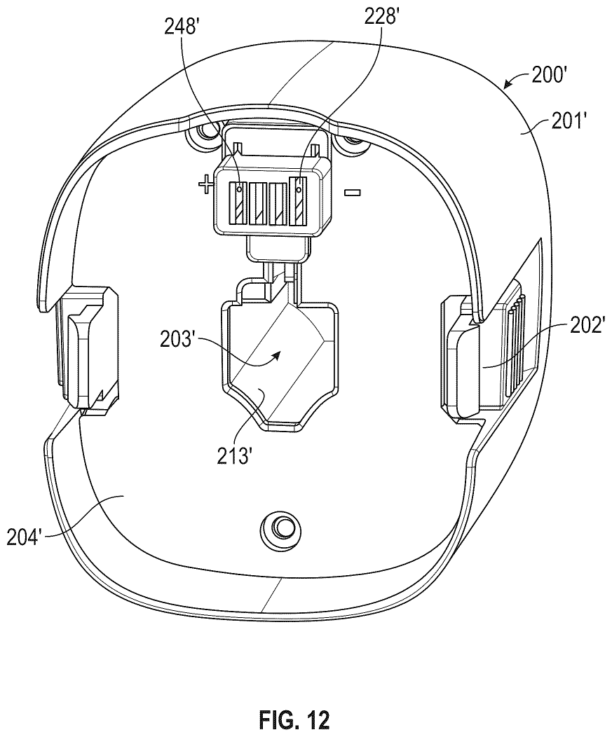

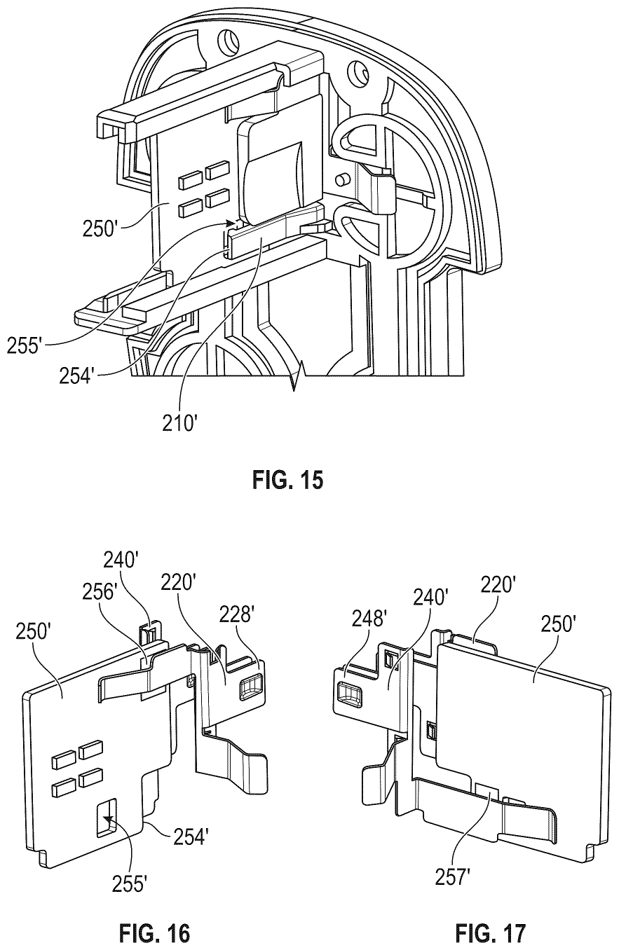

Referring now to FIGS. 8-18, an electromechanical, hand-held, powered surgical instrument 10′, in accordance with aspects of the disclosure includes a handle assembly 100′, an elongated shaft 300′ extending from the handle assembly 100′, and an end effector 400′ coupled to a distal end of the elongated shaft 300′. The end effector 400′ is configured for actuation and manipulation by the handle assembly 100′. Surgical instrument 10′ may be any battery powered instrument, handheld or robotic, such as a powered surgical stapler, grasper, cutter, electrosurgical device, etc.

Handle assembly 100′ includes a handle housing 101′, which houses various electronic component(s) 120′ (e.g., electronic component 120′ circuit board, motors, sensors, controllers, etc., FIG. 11) and mechanical components configured to control operation of the surgical instrument 10′. The surgical instrument 10′ additionally includes a battery assembly 200′ which is configured to removably couple to the handle assembly 100′ and supply power to the electronic components of the surgical instrument 10′. In aspects, the handle assembly 100′ includes a handle connector 102′ which is configured to releasably engage a battery connector 202′ formed by, or coupled to, a battery housing 201′ of the battery assembly 200′ to secure the battery assembly 200′ to the handle housing 101′. The handle connector 102′ and battery connector 202′ may be any suitable connection mechanism that enables releasable attachment of two components (e.g., press-clip, threading, etc.). In aspects, the handle assembly 100′ defines a handle finger 130′ configured to be positioned through an opening 203′ defined by a cover 204′ of the battery assembly 200′ and through a slot 213′ defined by the battery housing 201′ of the battery assembly 200′.

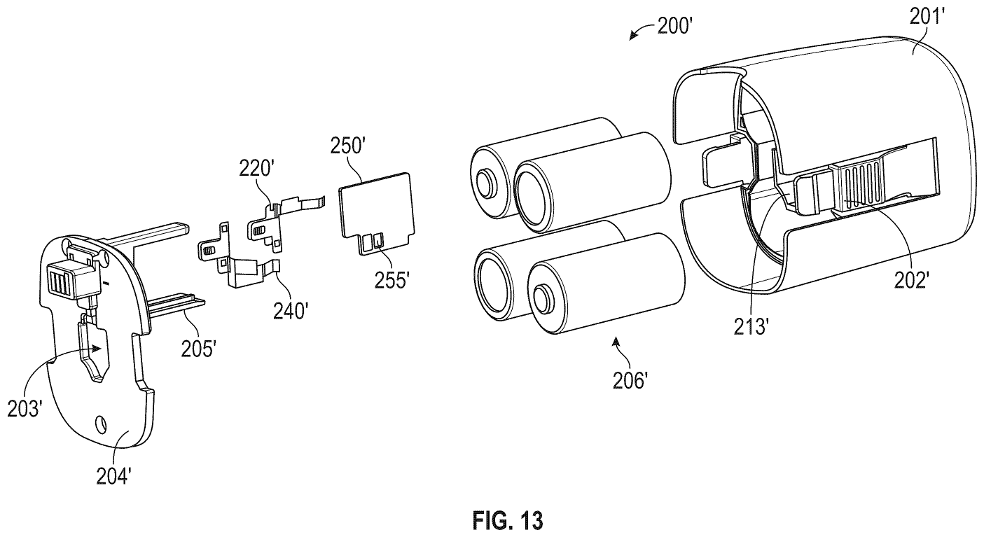

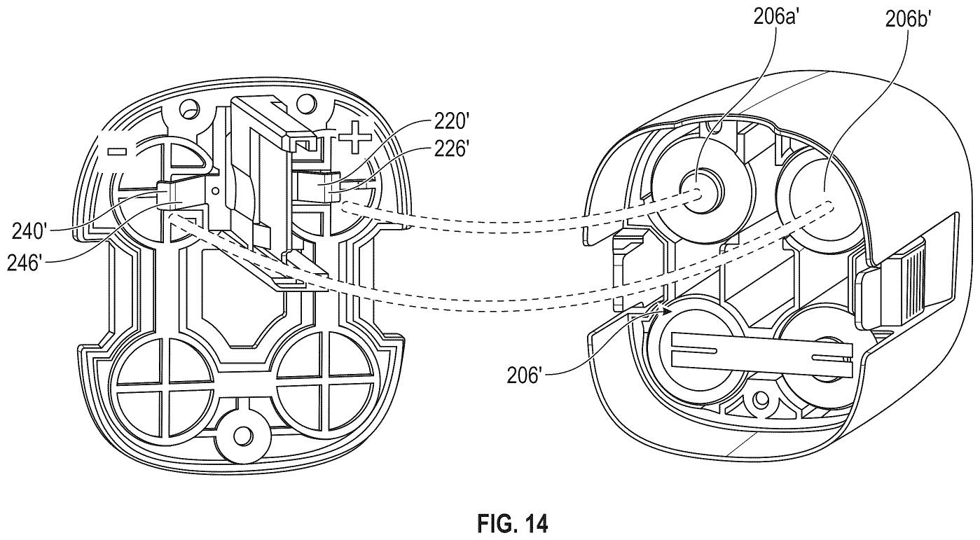

With reference to FIGS. 13 and 14, the battery assembly 200′ includes one or more battery cells 206′ disposed within the battery housing 201′ and enclosed within the battery housing 201′ by the cover 204′. The battery cell 206′ is configured to electrically couple to the electronic component 120′ of the surgical instrument 10′ when the battery assembly 200′ is coupled to the handle assembly 100′ to form a circuit therewith and provide power to the electrical components (e.g., the electronic component 120′, motors, sensors, etc.) of the surgical instrument 10′. The battery assembly 200′ also includes a discharge circuit board 250′ which is configured to discharge the battery cell 206′ after the battery cell 206′ is used with, and disconnected from, the surgical instrument 10′ (e.g., when the battery assembly 200′ is in a disposal state), as described in further detail below.

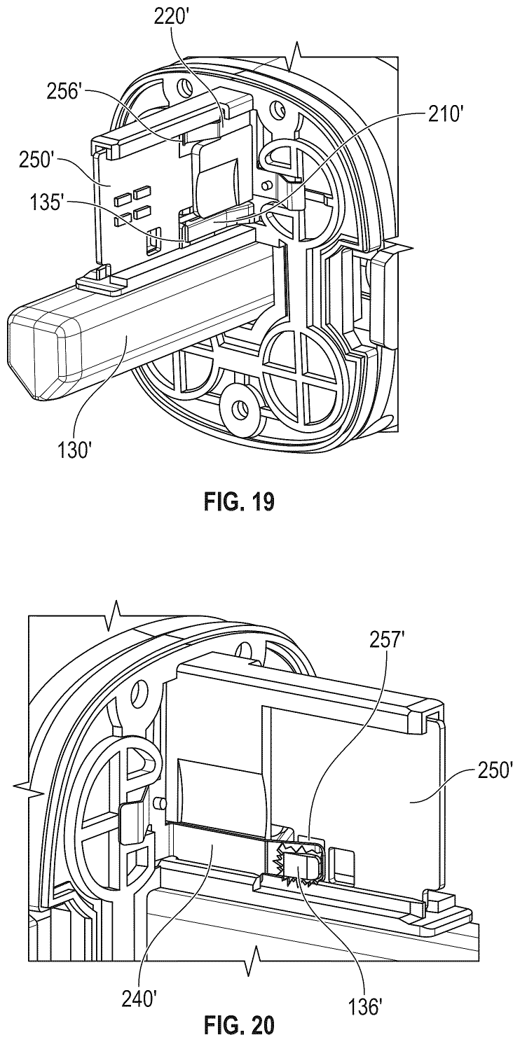

The discharge circuit board 250′ contains a passive resistive load which is conductively coupled to a first contact pad 256′ (FIG. 16) and a second contact pad 257′ (FIG. 17) and is movable within the battery housing 201′ between a first position (FIGS. 14, 15, and 18) and a second position (FIGS. 19-21). The discharge circuit board 250′ remains in the first position until the battery assembly 200′ is connected to the handle assembly 100′. The discharge circuit board 250′ is moved to the second position when the battery assembly 200′ is connected to the handle assembly 100′ and remains in the second position after the battery assembly 200′ is removed from the handle assembly 100′. The passive resistive load of the discharge circuit board 250′ may include one or more resistors. The first contact pad 256′ may be disposed on one side (e.g., underside) of the discharge circuit board 250′ and the second contact pad 257′ may be disposed on the other side (e.g., topside) of the discharge circuit board 250′. In an aspect, the cover 204′ defines a track 205′ within which the discharge circuit board 250′ is configured to longitudinally slide between the first position and the second position.

The battery assembly 200′ further includes a spring clip 210′ or any other suitable retaining device that is positioned relative to the discharge circuit board 250′ such that the spring clip 210′ maintains the position of the discharge circuit board 250′ in the first position prior to the battery assembly 200′ ever being coupled to any handle assembly 100′. The spring clip 210′ also maintains the discharge circuit board 250′ in the second position after the battery assembly 200′ is removed from the handle assembly 100′. In an aspect, the spring clip 210′ extends from the cover 204′ and a portion of the spring clip 210′ is positioned within an opening 255′ defined through the discharge circuit board 250′ to retain the discharge circuit board 250′ in the initial, first, position until a force is imparted upon the discharge circuit board 250′ to move the discharge circuit board 250′ along the track 205′ to the second position. When the discharge circuit board 250′ is moved to the second position, the spring clip 210′ rests against a distal surface 254′ of the print circuit board 250′ to retain the discharge circuit board 250′ in the second position, both while the battery assembly 200′ is coupled to the handle assembly 100′ and after the battery assembly 200′ is removed from the handle assembly 100′. Although described as being positioned within an opening 255′ and resting against a distal surface 254′, the spring clip 210′ may be configured in any manner suitable for retaining the discharge circuit board 250′ in the first and second positions. For example, the discharge circuit board 250′ may include one or more recesses (not shown) configured to receive the spring clip 210′, or other suitable configurations, to retain the discharge circuit board 250′ in one or more positions along the track 205′.

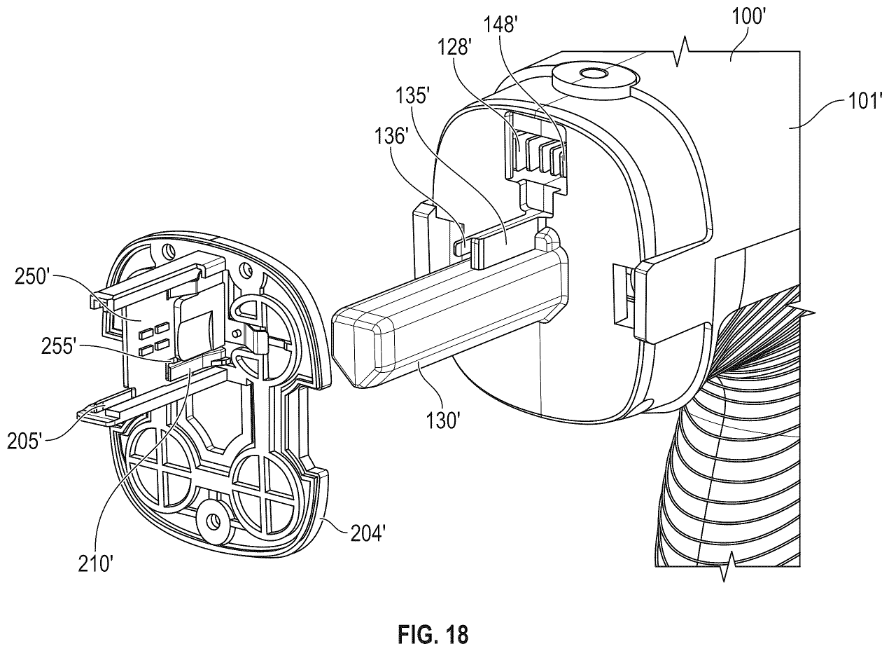

The battery assembly 200′ also includes a first battery contact 220′ and a second battery contact 240′ connected to the cover 204′ and positioned relative to the track 205′ in which the discharge circuit board 250′ is slidably disposed. In aspects, at least one of the first battery contact 220′ and the second battery contact 240′ is a leaf spring. The first battery contact 220′ and the second battery contact 240′ are fixed in position relative to the track 205′ of the cover 204′such that the discharge circuit board 250′ is movable relative to the first battery contact 220′ and the second battery contact 240′. The first battery contact 220′ includes a first battery cell contact 226′, that is electrically coupled to a first terminal 206a′ of the battery cell 206′, and a first instrument contact 228′ which is accessible through the cover 204′ to contact a first electrical terminal 128′ (FIG. 18) of the handle assembly 100′ when the battery assembly 200′ is coupled to the handle assembly 100′. Similarly, the second battery contact 240′ includes a second battery cell contact 246′, that is electrically coupled to a second terminal 206b′ of the battery cell 206′, and a second instrument contact 248′ which is accessible through the cover 204′ to contact a second electrical terminal 148′ (FIG. 18) of the handle assembly 100′ when the battery assembly 200′ is coupled to the handle assembly 100′. The first electrical terminal 128′ and the second electrical terminal 148′ are electrically coupled to the electronic component 120′ of the handle assembly 100′ and are configured to close the electrical circuit, thereby providing electrical power to the electronic components (e.g., electronic component 120) of the handle assembly 100′, when the battery assembly 200′ is coupled to the handle assembly 100′.

The handle assembly 100′ includes a handle finger 130′ extending from the handle housing 101′ and a first protrusion 135′ and a second protrusion 136′ extending from the handle finger 130′. The first protrusion 135′ is positioned such that during the process of coupling the battery assembly 200′ to the handle assembly 100′, the first protrusion 135′ engages the spring clip 210′, forcing the spring clip 210′ outward to release the discharge circuit board 250′ from the spring clip 210′, and engages the distal surface 254′ (FIG. 16) of the discharge circuit board 250′, moving the discharge circuit board 250′ along the track 205′ of the cover 204′ from the first position (FIG. 18) to the second position (FIG. 19) as the battery assembly 200′ is being connected to the handle assembly 100′. The second protrusion 136′ is positioned such that during the process of coupling the battery assembly 200′ to the handle assembly 100′, the second protrusion 136′ engages the second battery contact 240′ to physically separate and prevent the second battery contact 240′ from contacting the second contact pad 257′ by sliding between the second battery contact 240′ and the second contact pad 257′.

After the discharge circuit board 250′ is moved to the second position from the first position, and while the battery assembly 200′ is still connected to the handle housing 101′, the first protrusion 135′ rests against the spring clip 210′ to prevent the spring clip 210′ from engaging the discharge circuit board 250′. With specific reference to FIG. 19, when the discharge circuit board 250′ is in the second position and the battery assembly 200′ is connected to the handle assembly 100′, the first battery contact 220′ is aligned, and in conductive contact, with the first contact pad 256′.

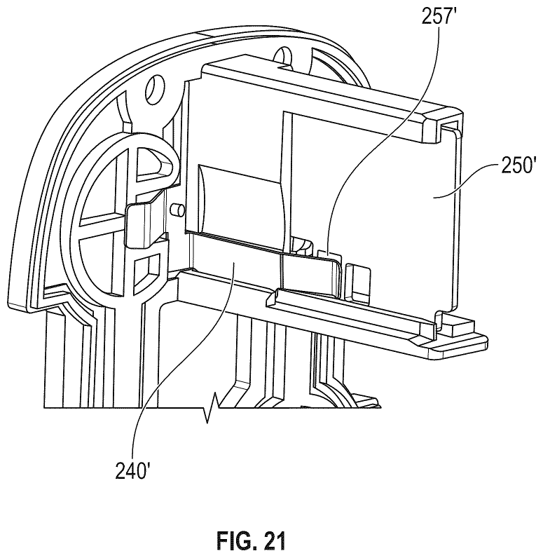

Additionally, with specific reference to FIG. 20, when the discharge circuit board 250′ is in the second position and the battery assembly 200′ is connected to the handle assembly 100′, the second battery contact 240′ is aligned, and not in conductive contact, with the second contact pad 257′. In particular, while the battery assembly 200′ is connected to the handle assembly 100′, the second protrusion 136′ separates the second battery contact 240′ from the second contact pad 257′, preventing electrical contact therebetween.

FIG. 21 illustrates the battery assembly 200′ in a disposal state. In particular, as illustrated in FIG. 21, subsequent to the battery assembly 200′ being removed from the handle assembly 100′, the discharge circuit board 250′ remains in the second position and is prevented from sliding to the first position by the abutment of the spring clip 210′ against the distal surface 254′ of the discharge circuit board 250′. With the battery assembly 200′ removed from the handle assembly 100′, and the discharge circuit board 250′ in the second position, the second protrusion 136′ is no longer disposed between the second battery contact 240′ and the second contact pad 257′ and therefore no longer physically separates the second battery contact 240′ from the second contact pad 257′. Thus, in this configuration, the second battery contact 240′ is electrically coupled to the second contact pad 257′, thereby closing the circuit between the battery cell 206′ and the resistive load of the discharge circuit board 250′. The resistive load of the discharge circuit board 250′ will discharge the battery cell 206′ until no more electrical energy is left in the battery cell 206′ of the battery assembly 200′.

The time to fully discharge the battery cell 206′ of the battery assembly 200′ depends on the capacity of the battery cell 206′ and the resistive load of the discharge circuit board 250′. In aspects, the resistive load of the discharge circuit board 250′ may be selectable, manually or automatically, to control the discharge rate. Resistors forming the resistive load of the discharge circuit board 250′ may be implemented in series and parallel to distribute electrical power as well as add safety redundancies to the discharging function of the discharge circuit board 250′.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

1. A surgical instrument comprising:
a handle assembly including:
a handle housing;
an electronic component disposed within the handle housing;
a handle finger extending from the handle housing; and
an electrical contact electrically coupled to the electronic component; and
a battery assembly configured to removably couple to the handle assembly, the battery assembly including:
a battery housing;
a battery cell disposed within the battery housing;
a battery contact electrically coupled to the battery cell; and
a discharge circuit board configured to discharge the battery cell and movable within the battery housing between a first position and a second position, wherein the discharge circuit board is moved from the first position to the second position by the handle finger upon coupling the battery assembly to the handle assembly.

2. The surgical instrument of claim 1, further comprising an elongated shaft extending from the handle assembly and an end effector operably coupled to the elongated shaft.

3. The surgical instrument of claim 1, wherein the battery assembly includes a spring clip configured to maintain the discharge circuit board in the second position after the battery assembly is removed from the handle assembly.

4. The surgical instrument of claim 3, wherein the spring clip is configured to maintain the discharge circuit board in the first position prior to the battery assembly being coupled to the handle assembly.

5. The surgical instrument of claim 1, wherein the battery contact is configured to conductively contact a contact pad of the discharge circuit board when the battery assembly is removed from the handle assembly.

6. The surgical instrument of claim 1, wherein the handle finger includes a protrusion configured to move a portion of the battery contact to block conductive contact between the battery contact and the discharge circuit board when the battery assembly is coupled to the handle assembly.

7. The surgical instrument of claim 1, wherein the battery contact is configured to conductively contact the electrical contact of the handle assembly when the battery assembly is coupled to the handle assembly to electrically couple the battery to the electronic component.

8. The surgical instrument of claim 1, wherein the handle finger defines an opening and the electrical contact extends through the opening.

9. The surgical instrument of claim 1, wherein the discharge circuit board is configured to longitudinally slide along a track of the battery assembly.

10. The surgical instrument of claim 1, wherein the battery housing includes a battery connector and the handle housing includes a handle connector configured to releasably mate with the battery connector.

11. The surgical instrument of claim 1, wherein the handle finger includes:
a first protrusion configured to move the discharge circuit board from the first position to the second position when the battery assembly is coupled to the handle assembly; and
a second protrusion configured to move a portion of the battery contact to block conductive contact between the battery contact and the discharge circuit board.

12. The surgical instrument of claim 1, wherein the battery contact is a first battery contact electrically coupled to a first terminal of the battery cell and configured to contact a first contact pad of the discharge circuit board when the discharge circuit board is in the second position, and wherein the battery assembly further includes a second battery contact electrically coupled to a second terminal of the battery cell and configured to contact a second contact pad of the discharge circuit board when the battery assembly is in the second position and in a discharge state.

13. A battery assembly for use with a surgical instrument, the battery assembly comprising:
a battery housing;
a battery cell disposed within the battery housing;
a battery contact electrically coupled to the battery cell; and
a discharge circuit board including a contact pad and movable within the battery housing between a first position in the battery housing and a second position in the battery housing, wherein the discharge circuit board is configured to discharge the battery cell when the contact pad is electrically coupled to the battery contact.

14. The battery assembly of claim 13, further comprising a spring clip configured to maintain the discharge circuit board in the first position and the second position.

15. The battery assembly of claim 13, further comprising a track defined in the battery housing, wherein the discharge circuit board is configured to longitudinally slide along the track.

16. The battery assembly of claim 13, wherein the battery contact is a second battery contact electrically coupled to a second terminal of the battery cell and configured to contact a second contact pad of the discharge circuit board when the discharge circuit board is in the second position and in a discharge state, and wherein the battery assembly further includes a first battery contact configured to contact a first contact pad of the discharge circuit board when the discharge circuit board is in the second position.

17. A surgical instrument comprising:
a handle assembly including:
a handle housing;
an electronic component disposed within the handle housing; and
an electrical contact electrically coupled to the electronic component; and
a battery assembly configured to removably couple to the handle assembly, the battery assembly including:
a battery housing;
a battery cell disposed within the battery housing;
a battery contact electrically coupled to the battery cell; and
a discharge circuit board including a contact pad and movable within the battery housing between a first position in the battery housing and a second position in the battery housing, wherein the discharge circuit board is configured to discharge the battery cell when the contact pad is electrically coupled to the battery contact.

18. The surgical instrument of claim 17, wherein the battery assembly includes a spring clip configured to maintain the discharge circuit board in the first position prior to the battery assembly being coupled to the handle assembly and maintain the discharge circuit board in the second position after the battery assembly is removed from the handle assembly.

19. The surgical instrument of claim 17, wherein the battery contact is configured to conductively contact a contact pad of the discharge circuit board when the battery assembly is removed from the handle assembly.

20. The surgical instrument of claim 17, wherein the handle assembly includes a handle finger and a protrusion extending from the handle finger, the protrusion configured to move a portion of the battery contact to block conductive contact between the battery contact and the discharge circuit board when the battery assembly is coupled to the handle assembly.

21-25. (canceled)
